(19) United States
(12) Patent Application Publication
(10) Pub. No.: US 2014/0209701 A1
(43) Pub. Date: Jul. 31, 2014

(54) Passive Diffuser Having a Non-Planar Wick

(71) Applicant: BATH & BODY WORKS BRAND MANAGEMENT, INC., Reynoldsburg, OH (US)

(72) Inventors: Raymond Cen, New Albany, OH (US); Heather Mastny, Dublin, OH (US); Noelle Border, Powell, OH (US); Mike Regina, Ramsey, NJ (US); Maureen Stanley, Blacklick, OH (US); Robert Wong, Jericho, NY (US)

(73) Assignee: BATH & BODY WORKS BRAND MANAGEMENT, INC., Reynoldsburg, OH (US)

(21) Appl. No.: 13/755,765

(22) Filed: Jan. 31, 2013

(51) Int. Cl. A61L 9/12

(52) U.S. Cl. CPC A61L 9/127
USPC 239/44

(57) ABSTRACT

A device for passively diffusing a liquid to the air of a surrounding area. A reservoir holds the liquid, the reservoir having a top end with an aperture therethrough. A non-planar wick extends through the aperture such that a lower end of the wick remains in contact with the liquid in the reservoir and an upper end of the wick is exposed to the surrounding area. The wick is formed of a fibrous, porous sheet of material having a top edge and a bottom edge of substantially the same width. The sheet of material is gradually gathered along a length of the sheet of material such that the upper end of the wick is less gathered than the lower end of the wick and the wick is self-supported in an upright position within the aperture.

### FIELD

The present disclosure relates to devices that passively diffuse liquids into the air of a surrounding area.

### BACKGROUND

U.S. Patent Application Publication No. 2012/0091221 discloses a wick diffuser employing a cellulosic based wick. The wick diffuser includes a base from which first and second arcuate panels upwardly and outwardly extend and flanking a center cellulosic wick. The wick is substantially planar in shape and upwardly extends from a container of volatile organic compound housed within the base. The base may be manufactured from a polymeric material or include a polymeric insert provided within a wooden surround. The diffuser has numerous spill resistance features as well as a low insertion force needed by the user to install the wick.

U.S. Pat. Nos. 7,252,244 and 7,469,842 disclose a volatile dispenser, including a reservoir containing a quantity of volatile liquid having a viscosity of no greater than 3.9 centipoises. Each of its individual components has a vapor pressure of no less than 0.008 mm Hg. The dispenser also includes an emanator fluidly coupled to the reservoir (either directly or through a wick) that is comprised of elongated oriented bonded fibers, the fibers being impermeable to the volatile material.

U.S. Pat. Nos. 6,708,897 and 6,921,025 disclose a unit for the transfer and distribution of the liquid using capillary action having an elongated shaft and a screen. The shaft includes a first capillary medium suitable for drawing the liquid from a reservoir into the shaft when a bottom portion of the shaft is disposed in the liquid. The screen is connected to the shaft and includes a second capillary medium suitable for receiving the liquid drawn by the first capillary medium and distributing it over at least a major part of the screen where it evaporates.

### SUMMARY

One aspect of the present application includes a device for passively diffusing a liquid to the air of a surrounding area. The device includes a reservoir holding the liquid, the reservoir having a top end with an aperture therethrough. The device also has a non-planar wick extending through the aperture such that a lower end of the wick remains in contact with the liquid in the reservoir and an upper end of the wick is exposed to the surrounding area. The wick is formed of a fibrous, porous sheet of material having a top edge and a bottom edge of substantially the same width. The sheet of material is gradually gathered along a length of the sheet of material such that the upper end of the wick is less gathered than the lower end of the wick and the wick is self-supported in an upright position within the aperture.

In another aspect of the present application, a device for passively diffusing a liquid to the air of a surrounding area includes a reservoir holding the liquid, the reservoir having a top end with an aperture therethrough. The device also has a non-planar wick extending through the aperture such that a lower end of the wick remains in contact with the liquid in the reservoir and an upper end of the wick is exposed to the surrounding area. A collar is coupled to the reservoir and surrounds the wick within the aperture. The collar provides a liquid-tight connection between the wick and reservoir.

In another aspect of the present application, a device for passively diffusing a liquid to the air of a surrounding area includes a reservoir holding the liquid, the reservoir having a top end with an aperture therethrough. A non-planar wick extends through the aperture such that a lower end of the wick remains in contact with the liquid in the reservoir and an upper end of the wick is exposed to the surrounding area. A collar is coupled to the reservoir and surrounds the wick within the aperture, so as to provide a liquid-tight connection between the wick and the reservoir. A screen surrounds the reservoir and the wick, the screen having a base supporting the reservoir and side walls extending upwardly from the base and shielding the wick. The wick is pleated along a plurality of longitudinally extending bend lines, two adjacent bend lines defining a longitudinally extending wall segment of the wick. The lower end of the wick is gathered such that adjacent wall segments face one another in substantially parallel relationship, and the upper end of the wick is splayed out such that adjacent wall segments extend at an angle to one another.

### BRIEF DESCRIPTION OF DRAWINGS

Examples of a device for passively diffusing a liquid to the air of a surrounding area are described with reference to the following Figures. The same numbers are used throughout the Figures to reference like features and like components.

FIG. 1 shows a perspective view of a device for passively diffusing a liquid to the air of a surrounding area of the present application.

FIG. 2 shows a side, cut-away view of the device of FIG. 1.

FIG. 3 shows a side, partially cut-away view of the device of FIGS. 1 and 2.

FIG. 4 shows an embodiment of a sheet of material for forming a wick of the device of the present application.

FIG. 5 shows an additional embodiment of a sheet of material.

FIG. 6 shows an additional embodiment of a sheet of material.

FIG. 7 shows the sheet of material of FIG. 6 assembled into a wick.

FIG. 8 shows an additional embodiment of a sheet of material.

FIG. 9 shows the sheet of material of FIG. 8 assembled into a wick.

FIG. 10 shows a perspective view of an embodiment of a reservoir of the device.

FIG. 11 shows an underside perspective view of a cap for the reservoir of FIG. 10.

FIG. 12 shows a perspective view of an embodiment of a collar of the device.

FIG. 13 shows an underside perspective view of the collar of FIG. 12.

FIG. 14 shows a front, cut-away view of the collar taken along section line A-A of FIG. 12.

FIG. 15 shows a side, cut-away view of the collar taken along section line B-B of FIG. 12.

FIG. 16 shows a top view of the collar of FIG. 12.

FIG. 17 shows a bottom view of the collar of FIG. 12.

FIG. 18 shows a perspective view of an embodiment of a screen for the device.

FIG. 19 shows a side view of the screen of FIG. 18.

FIG. 20 shows a front view of the screen of FIG. 18.

FIG. 21 shows a front view of the device including an additional embodiment of the screen.

### DETAILED DESCRIPTION OF DRAWINGS

In the present description, certain terms have been used for brevity, clearness and understanding. No unnecessary limitations are to be implied therefrom beyond the requirement of the prior art because such terms are used for descriptive purposes only and are intended to be broadly construed. The different devices described herein may be used alone or in combination with other devices. Various equivalents, alternatives, and modifications are possible within the scope of the appended claims. Each limitation in the appended claims is intended to invoke interpretation under 35 USC §112, sixth paragraph, only if the terms “means for” or “step for” are explicitly recited in the respective limitation.

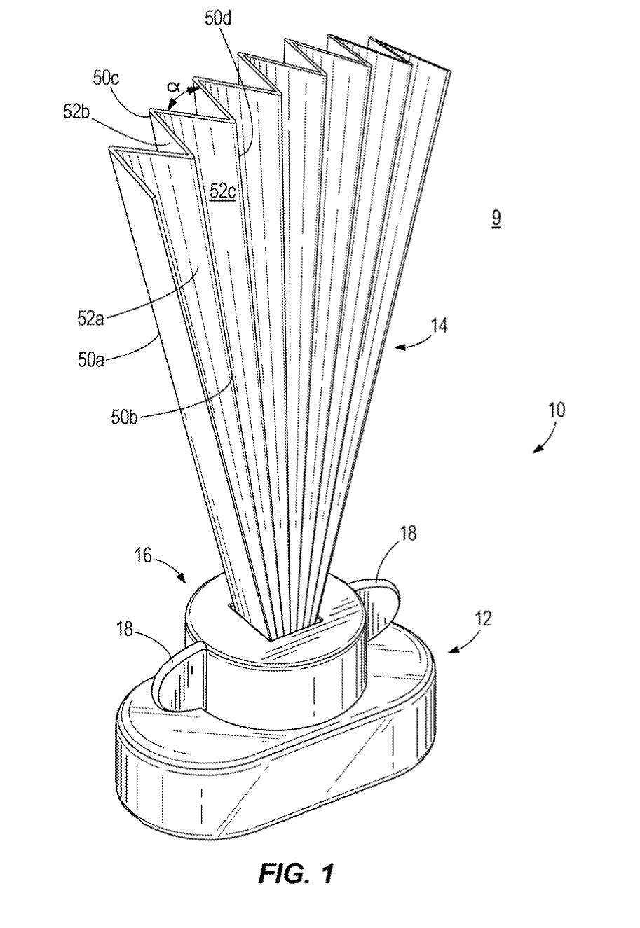

FIG. 1 illustrates a device 10 for passively diffusing a liquid to the air of a surrounding area 9. Thus surrounding area 9 can be, for example, a room or a living space. A reservoir 12 holds the liquid. A non-planar wick 14 extends from the reservoir 12 through a collar 16. Collar 16 has wings 18 located on opposite sides of the collar 16 for providing a visual indication to a consumer that the collar 16 is correctly positioned atop the reservoir 12, as will be further described herein below.

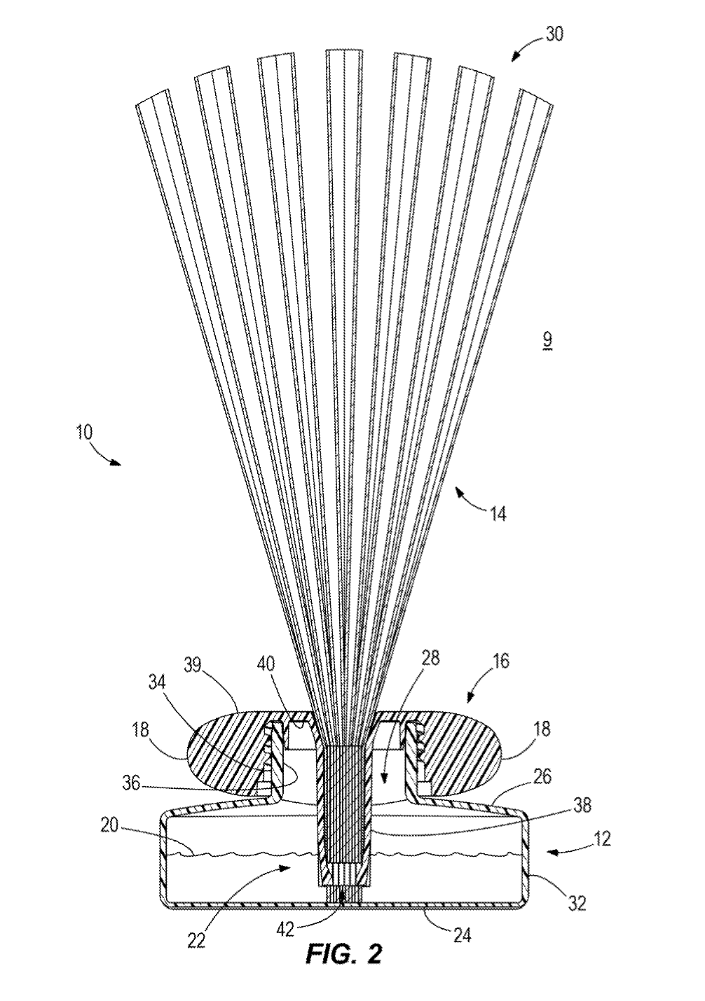

FIG. 2 shows the device 10 as a front, cut-away view. The reservoir 12 contains a liquid 20. A lower end 22 of the wick 14 remains in contact with the liquid 20 within the reservoir 12. To do so, the lower end 22 of the wick 14 may be positioned adjacent a bottom end 24 of the reservoir 12. A top end 26 of the reservoir 12 has an aperture 28 therethrough. The wick 14 extends through the aperture 28 such that an upper end 30 of the wick 14 is exposed to the surrounding area 9, while the lower end 22 of the wick 14 contacts the liquid 20 in the reservoir 12.

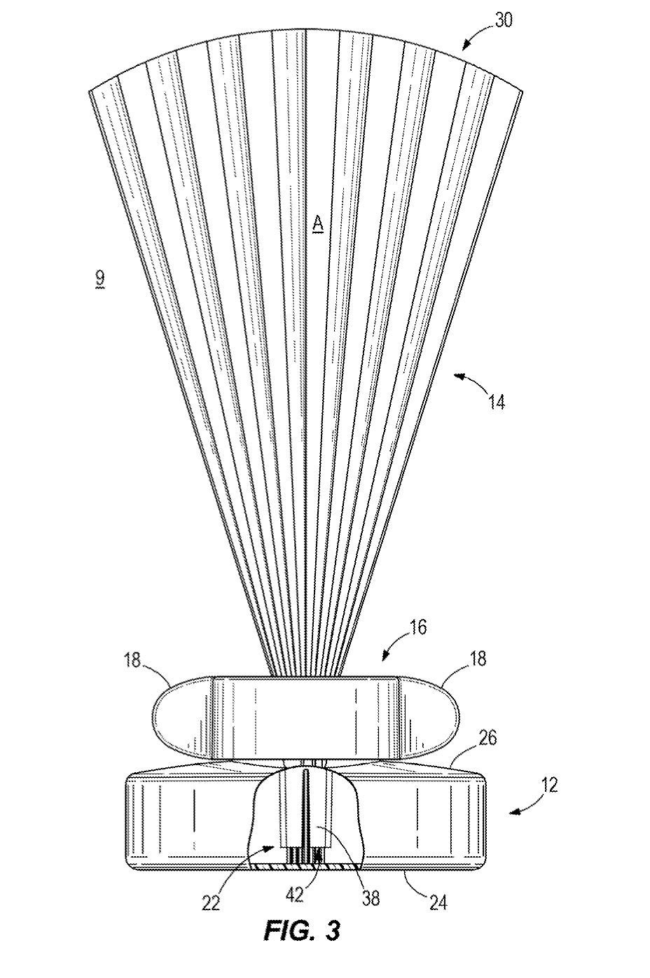

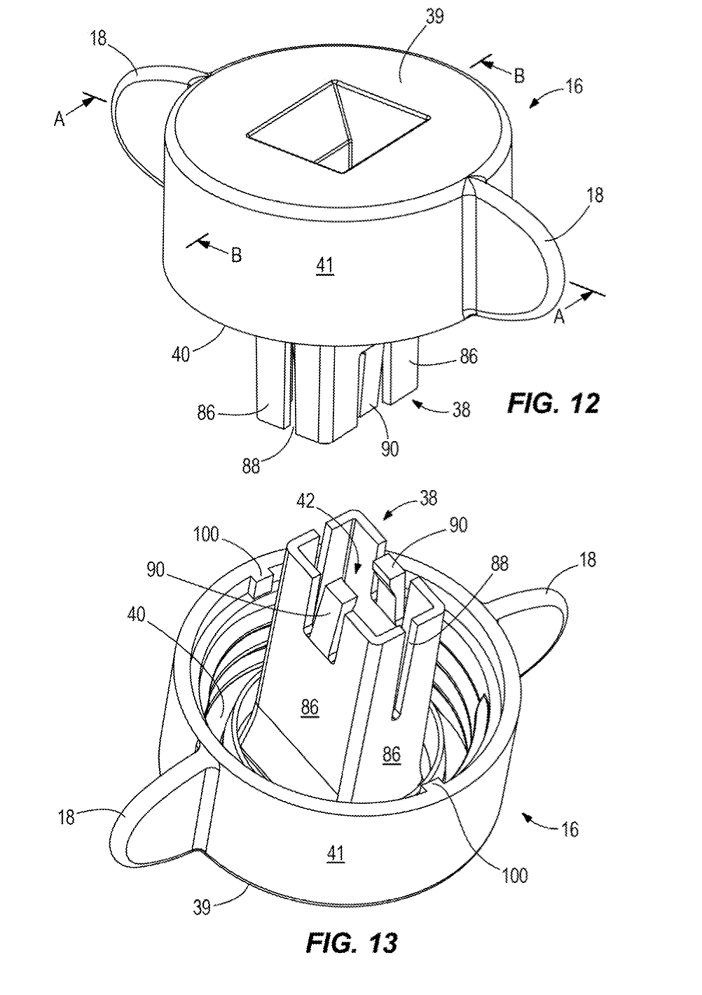

The reservoir 12 comprises a main body 32 and an upwardly extending neck 34. (See also FIG. 10.) An inner surface 36 of the neck 34 further defines the aperture 28. The collar 16 is coupled to the neck 34 and surrounds the wick 14 within the aperture 28 so as to provide a liquid-tight connection between the wick 14 and the reservoir 12. The collar 16 has an upper side 39 and a lower side 40. The wings 18 extend from an outer edge 41 (see FIG. 12) of the collar 16. The collar 16 has a sleeve 38 (see also FIGS. 3 and 13) that extends downwardly from the lower side 40 of the collar 16 and through the aperture 28. The sleeve 38 has an insertion opening 42 therethrough that is configured to receive the lower end 22 of the wick 14. (See also FIGS. 12 and 13.) FIG. 3 shows a front, cut-away view into the reservoir 12, such that the sleeve 38 is more fully shown.

In order that the liquid 20 diffuses to the air of the surrounding area 9, the liquid travels from the lower end 22 of the wick 14 to the upper end 30 of the wick 14 via capillary action. Where the wick 14 is exposed to the air of the surrounding area 9, the liquid 20 evaporates, i.e. changes from liquid to gaseous form, and thereafter diffuses throughout the air of the surrounding area 9. The liquid can be, for example, a volatile or nonvolatile liquid. If the liquid 20 is a volatile liquid, the liquid 20 can be a volatile organic compound (VOC). In one example, the liquid 20 is a volatile organic compound having a scent. In one example, the volatile organic compound has a fragrance range of 45-80% and a dipropylene gycol methyl ether acetate (DPMA) range of 20-55%.

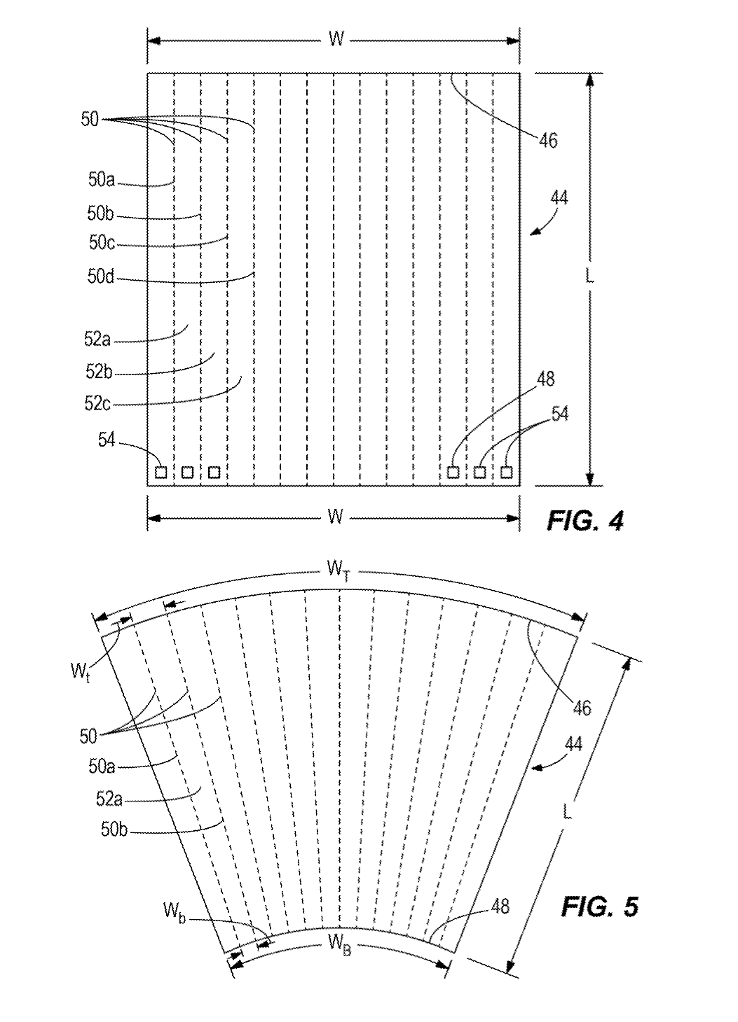

The wick 14 is formed from a sheet of material 44 (see FIGS. 4-6 and 8). The sheet of material 44 can be a blotter paper, a hydrophobic non-woven, or a laminated structure. The sheet of material 44 can be, for example, construction paper; Orlandi blotter paper (available from Orlandi, Inc. of Farmingdale, N.Y.); 4DG felt (available from New England Nonwovens, LLC of Franklin, Mass.); and/or thermally bonded carded nonwovens or flat and point-bonded nonwovens having a weight range of 17-120 grams/meter squared and a thickness of 0.6-20 millimeters and made of fibers such as polyester, aramid, PPS, P-84, viscose rayon, polypropylene, and blends thereof (available from Bondex, Inc. of Trenton, S.C.). It should be understood to those having ordinary skill in the art that the sheet of paper 44 from which the wick 14 is formed can be made of any fibrous porous paper-like materials or any non-porous fiber nonwovens. In other words, any material that is capable of wicking liquid 20 from the lower end 22 of the wick 14 to the upper end 30 of the wick 14 is suitable for the present application. The sheet of material 44 can also be semi-rigid so as to contribute to the self-supporting nature of the wick 14, allowing the wick 14 to support its own shape and weight, as well as the weight of any liquid 20 that the wick 14 has sucked from the reservoir 12. The caliper (pt) chosen for the sheet of material 44 will affect the absorption capabilities and likelihood of deformation after folding. In one embodiment the sheet of material 44 has a caliper that ranges from about 9-17pt. For example, the sheet of material 44 can be 12pt or 17pt Orlandi blotter paper.

Now with reference to FIG. 4, the sheet of material 44 from which the wick 14 is formed will be further described. The wick 14 is formed of a sheet of material 44 having a top edge 46 and a bottom edge 48 of substantially the same width W. In one embodiment, for example, the width W of the top and bottom edges 46, 48 is the same, while in other embodiments, the width of the bottom edge 48 ranges from 30%-200% of the width of the top edge 46. The sheet of material 44 may be provided with cut outs 54, the purpose of which will be described further herein below. To form the wick 14 from the sheet of material 44, the wick 14 is folded, or pleated, along a plurality of longitudinally extending bend lines 50. This is done by folding the sheet of material 44 accordion style, i.e. back and forth in opposite directions, at each bend line 50. As shown in FIG. 1, once pleated, two adjacent bend lines, such as bend lines 50a and 50b, define a longitudinally extending wall segment 52a of the wick 14. Similarly, bend lines 50b and 50c define longitudinally extending wall segment 52b and bend lines 50c and 50d define longitudinally extending wall segment 52c. With reference to each of FIGS. 1-3, the lower end 22 of the wick 14 is then gathered such that adjacent wall segments 52a, 52b, 52c face one another in substantially parallel relationship, as shown by the lower end 22 of the wick 14 in FIG. 2. The upper end 30 of the wick 14 is splayed out such that adjacent wall segments 52a, 52b, 52c extend at an angle to one another, for example, angle α in FIG. 1. In other words, once pleated, the sheet of material 44 is gradually gathered along its length L such that the upper end 30 of the wick 14 is less gathered than the lower end 22 of the wick 14.

This pleated nature of the wick 14 provides several advantages. For example, once it has been pleated, the sheet of material 44 can support itself in an upright position within the aperture 28 of the reservoir 12, as the pleated nature of the wick 14 provides the wick 14 with structural rigidity and integrity. Such structural integrity reduces the need for complex plastic parts to hold the wick 14 in an upright position. Further, the longitudinally extending wall segments 52a, 52b, 52c, etc. provide the wick 14 with a diffusing-capable surface area of W×L that is greater than a planform surface area A (see FIG. 3) of the wick 14. This provides the wick 14 with a relatively large diffusing-capable surface area (in comparison to its planform surface area A) for diffusing fragrance 20 to the surrounding area 9. In one example, pleating the wick 14 provides a 500% increase over a non-pleated planar wick with the same planform surface area A. The pleated nature of the wick 14 also allows a consumer to adjust the angle α between the longitudinally extending wall segments 52 so as to adjust the rate of evaporation of liquid 20 from the upper end 30 of the wick 14. Further, because the lower end 22 of the wick 14 is gathered such that the full width W of the lower edge 48 of the sheet of paper 44 is in contact with the liquid 20, there is more porous mass in the dipping portion (lower end 22) of the wick 14 which in turn provides improved capillary suction capabilities. This is turn positively affects the evaporation profile of the wick 14 and the fragrance release rate as a function of the material of which the wick 14 is made and the size of the wick 14. Further, the wick 14 has a seamless transition from the dipping portion at the lower end 22 of the wick 14 to the exposed portion at the upper end 30 of the wick 14. In other words, the evaporation profile and fragrance release rate of the wick 14 are not limited by the size of the dipping portion (lower end 22) of the wick 14, because the entire width W of the sheet of material 44 is dipped into the liquid 20. Capillary suction within the dipping portion is therefore not a rate limiting factor with the wick 14 disclosed herein.

In one embodiment, the wick 14 has a total surface area (double sided) from 20-100 square inches. In one embodiment, the width of a longitudinally extending wall segment 52 ranges from 0.1-2 inches, preferably from 0.2-1 inch, and more preferably from 3/16-¾ of an inch. When made from 12pt or 17pt blotter paper, for example blotter paper provided by Orlandi of Farmingdale, N.Y., a wick 14 with these dimensions is capable of consuming fragrance at a rate from 0.2-2 grams/day and can deliver fragrance to the surrounding area 9 for at least seven days after complete consumption of liquid 20 from within the reservoir 12. Such a wick 14 is not sensitive to the type of fragrance used, and the fragrance need not include a significant amount of top notes.

FIG. 5 shows another embodiment of a sheet of material 44 for forming the wick 14. The sheet of material 44 has a top edge 46 and a bottom edge 48. Here however, the sheet of material 44 has a width W_{B} at its bottom edge 48 that is not substantially the same as the width W_{T} at the top edge 46. This is because the sheet of material 44 has a curved trapezoidal shape, wherein the width W_{B} of the bottom edge 48 gradually widens to the width W_{T} at the top edge 46. Once pleated and gathered, a wick 14 formed from a sheet of material 44 as shown in FIG. 5 has a thinner gathered lower end 22 than the gathered lower end 22 of a wick 14 formed from the sheet of material 44 of FIG. 4. The lower end 22 is therefore relatively easier to insert into the insertion opening 42 of the sleeve 38. Further, the gathered lower end 22 of the wick 14 (despite having less surface area than the lower end 22 of a wick 14 formed from the sheet of material 44 shown in FIG. 4) is still able to support a relatively large surface area at its upper end 30. In one embodiment, the longitudinally extending wall segment 52a has a bottom width W_{b} of 5/16 of an inch, and widens to a top width Wₜ of 11/16 of an inch. In one embodiment, the length L of the longitudinally extending wall segment 52a is 6.5 inches.

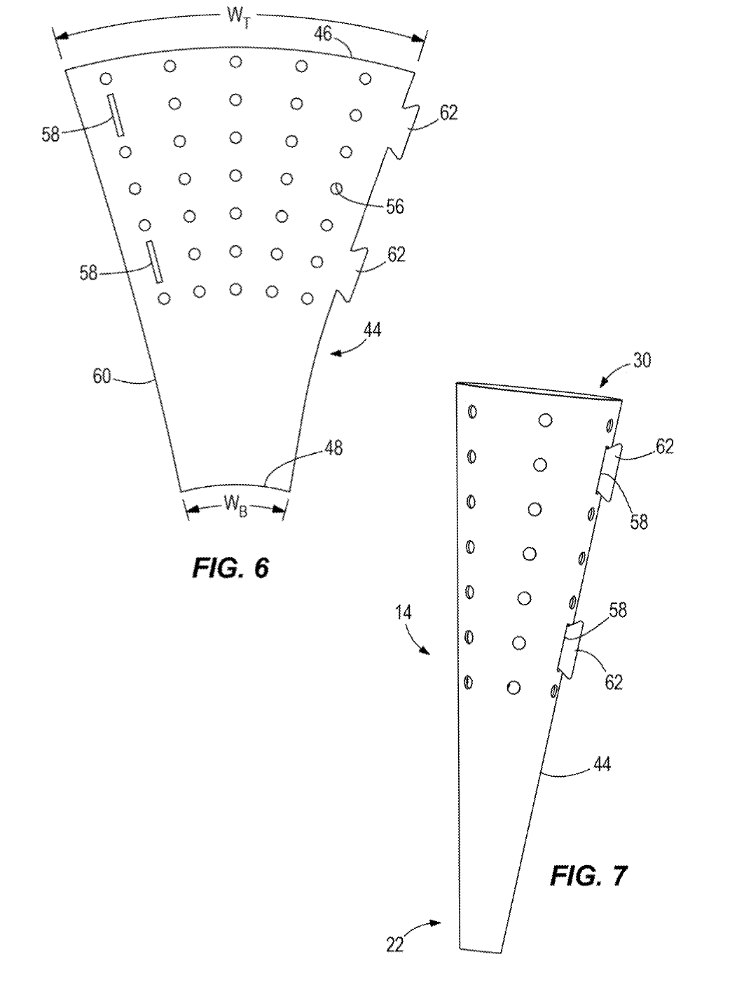

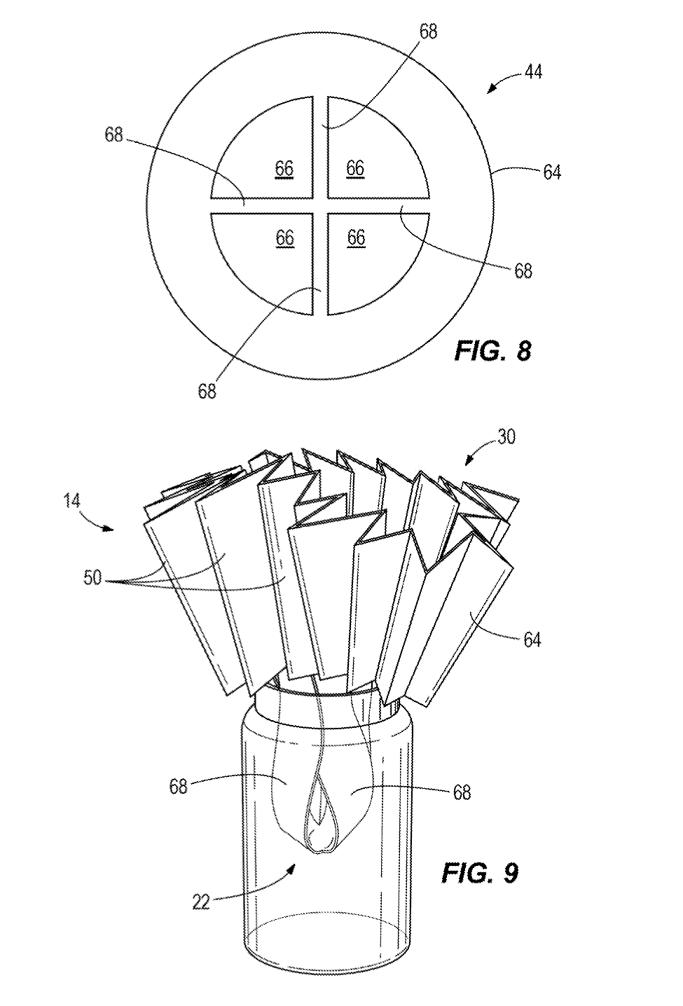

Alternative embodiments of the sheet of material 44 for forming the wick 14 are contemplated within the scope of the present disclosure. As shown in FIGS. 6 and 8, the sheet of material 44 may take shapes other than those shown in FIGS. 4 and 5. For example, in FIG. 6, the sheet of material 44 has a top edge 46 and a bottom edge 48 much like the sheet of material 44 shown in FIG. 5, i.e., the bottom edge 48 has a width W_{B} that is less than the width W_{T} of the top edge 46. In FIG. 6 however, the width W_{B} is more pronounceably narrower than the width W_{T} of the top edge 46 when compared to FIG. 5. Further, the sheet of material 44 can be provided with holes 56 therein. The holes 56 can provide increased air conduction, so as to provide an even evaporation rate. The sheet of material 44 can also be provided with slots 58. The slots 58 are located adjacent a side edge 60 of the sheet of material 44. Tabs 62 are provided on the opposite side edge 60 of the sheet of material 44. As shown in FIG. 7, in order to form the wick 14 from the sheet of material 44 of FIG. 6, the tabs 62 are inserted into the slots 58 after the sheet of material 44 has been rolled into a cone shape. A lower end of the cone forms the lower end 22 of the wick 14. This lower end 22 contacts the liquid 20 within the reservoir 12. The wick 14 therefore has a non-planar cone shape that provides a large dipping portion, a large diffusing-capable surface area, and self-supporting rigidity to the wick 14.

As shown in FIG. 8, the sheet of material 44 may also have a circular shape in an unfolded form. For example, the sheet of material 44 can comprise a circle 64 with pie-shaped cut outs 66 therein. Cross bars 68 connect an outer edge of the circle 64. To form the wick 14, the sheet of material 44 is folded such that the circle 64 is pleated along a plurality of bend lines 50 and the cross bars 68 are intertwined with one another. The folded, intertwined cross bars 68 form the lower end 22 of the wick 14, for insertion into the liquid 20 in the reservoir 12. The upper end 30 of the wick 14 is formed by the pleated circle 64 portion of the sheet of material 44. The wick 14 therefore has a non-planar polyhedron shape that provides a large dipping portion, a large diffusing-capable surface area, and self-supporting rigidity to the wick 14.

The wick 14 can have many other shapes other than those shown in the Figures. For example, any three dimensional shape will provide the objectives stated hereinabove of creating a rigid, self-supporting wick with a large dipping portion and a large diffusing-capable surface area. For example, the wick 14 could have the shape of a polyhedron or could be folded by origami. Further, the wick 14 could include more than one material, such as a combination of felt and paper sewn, glued, or stapled together. The felt provides the wick 14 with capillary suction, while the paper provides structural rigidity and shape to the wick 14. The wick 14 can also comprise reed, plastic, or wood supports that provide extra rigidity to the shape of the wick. Any of the wicks shown in FIGS. 1-8 can be provided with holes 56 (see FIGS. 6 and 7) to increase air conduction near the upper end 30 of the wick 14.

Now with reference to FIG. 10, the reservoir 12 will be further described. As described hereinabove, the reservoir 12 has a bottom end 24, a top end 26 and an aperture 28 providing access to the reservoir 12. The top end 26 of the reservoir 12 has a neck 34 extending therefrom. An inner surface 36 of the neck 34 defines the aperture 28. The neck 34 has an outer thread 70, the purpose of which will be further described herein below. The neck 34 also has an alignment portion 72. The alignment portion 72 comprises a detent 74 and a rounded nub 76 separated by a slot 78. The purpose and function of the detent 74, nub 76, and slot 78 will be further described herein below.

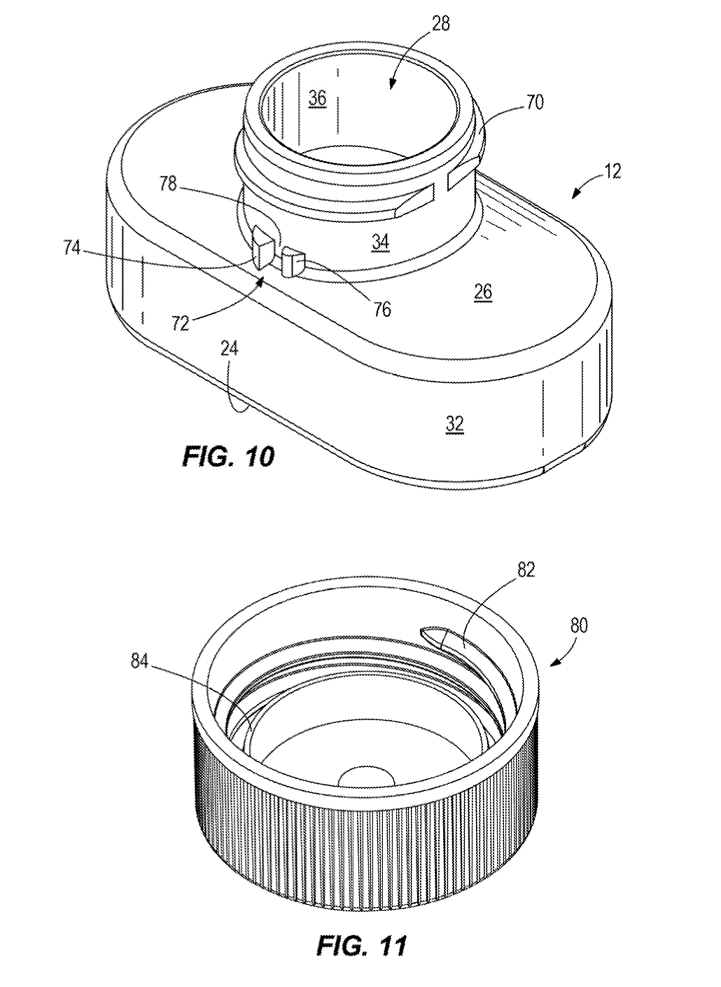

FIG. 11 shows an underside of a cap 80 that fits over the neck 34 of the reservoir 12 when the device 10 is not in use, such as when the device 10 is being shipped from one place to another. The cap 80 comprises an inner thread 82. The inner thread 82 engages with the outer thread 70 on the neck 34 of the reservoir 12 to hold the cap 80 in place on the neck 34 when the device 10 is being shipped. Cap 80 also comprises an annular projecting portion 84 that fits within the neck 34 and contacts the inner surface 36 of the neck, so as to provide a liquid-tight fit with the neck 34 while the device 10 is being shipped.

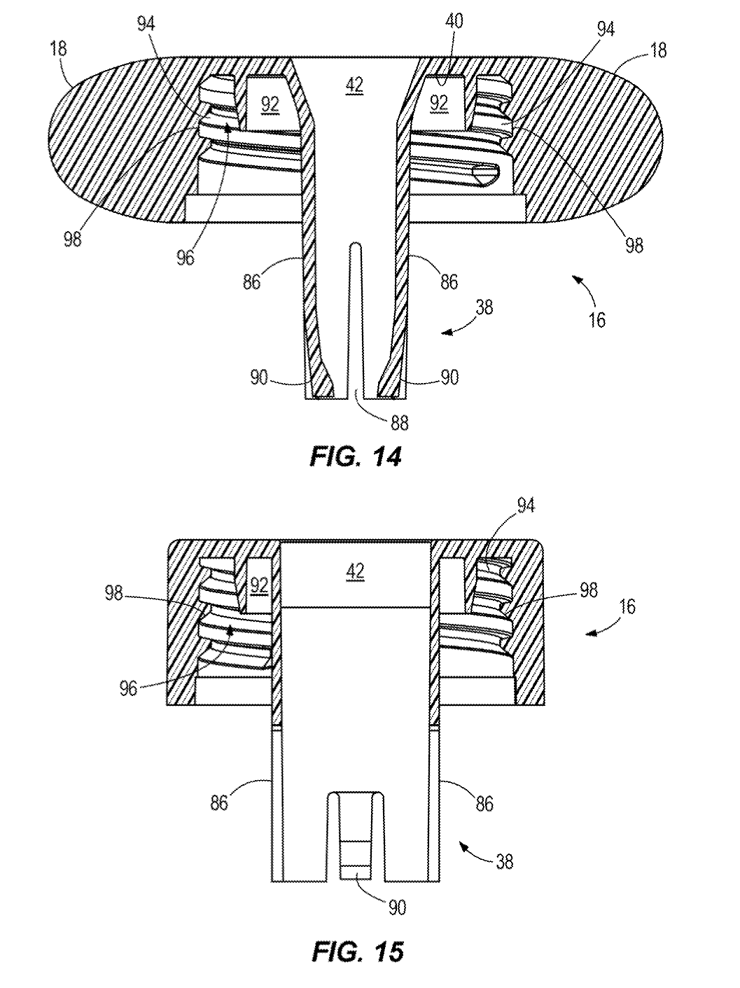

Now with reference to FIGS. 12 and 13, the collar 16 will be further described. As described herein above, the collar 16 has an upper side 39 and a lower side 40. Wings 18 are provided on an outer edge 41 of the collar 16. A sleeve 38 extends downwardly from the lower side 40 of the collar 16. The sleeve 38 has several side walls 86 that define an insertion opening 42. The side walls 86 may be provided with slits 88 that allow the liquid 20 to contact the lower end 22 of the wick 14. In one embodiment, opposite side walls 86 may be provided with radially inwardly extending elastic fingers 90. The fingers 90 secure the lower end 22 of the wick 14 within the insertion opening 42 by applying a radially inwardly directed force on the lower end 22 of the wick 14. The fingers 90 may also engage with the cutouts 54 (FIG. 4) in the lower end 22 of the wick 14 to further secure the wick 14 in place within the insertion opening 42. The wick 14 is secured in the insertion opening 42 such that a liquid-tight connection is formed between the wick 14 and the collar 16. If the device 10 is temporarily tipped, liquid 20 will not leak from the collar 16, but instead will be soaked up by the wick 14. Further views of the collar 16 are provided in FIGS. 14 and 15, where FIG. 14 is a sectional view along A-A of FIG. 12 and FIG. 15 is a sectional view along B-B.

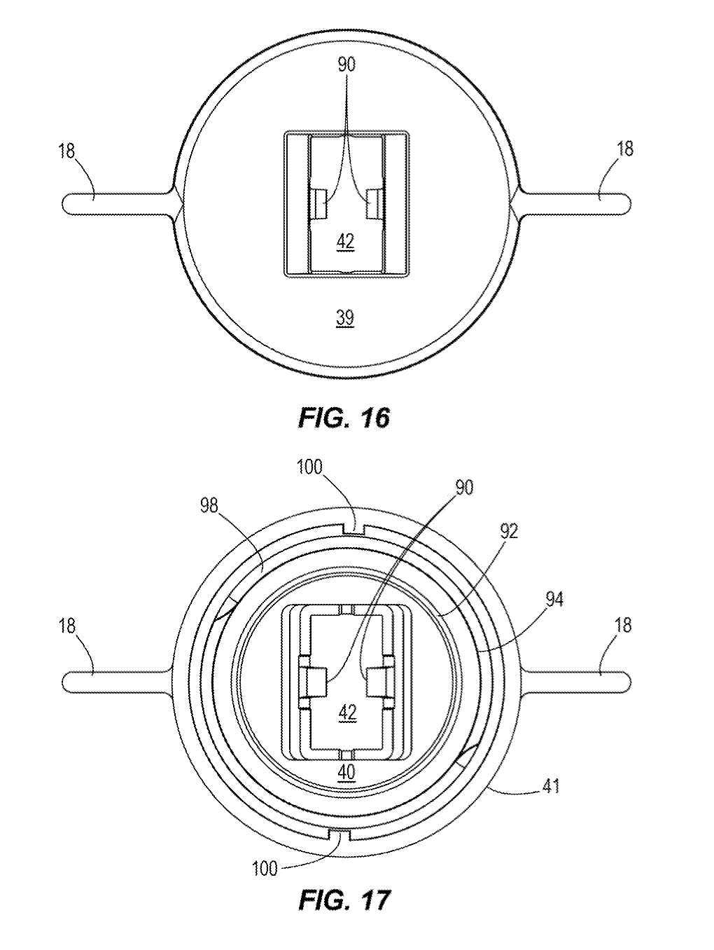

Now with reference to FIGS. 14-17, the collar 16 will be even further described. The collar 16 comprises an inner annulus 92 extending downwardly from the lower side 40 of the collar 16. An outer annulus 94 also extends downwardly from the lower side 40 of the collar 16. The inner annulus 92 and outer annulus 94 are located radially outwardly of the sleeve 38 and have a recess 96 therebetween for insertion of the neck 34 of the reservoir 12 (see FIG. 2). The outer annulus 94 of the collar 16 has an inner thread 98 that engages with the outer thread 70 on the neck 34 to form a threaded connection between the collar 16 and the neck 34. During assembly of the collar 16 to the reservoir 12, after the neck 34 has been inserted into the recess 96, engagement of the outer thread 70 with the inner thread 98 provides a liquid-tight fit between the reservoir 12 and the collar 16. This in turn provides a liquid-tight connection between the reservoir 12 and the wick 14 as the wick 14 is secured in the insertion opening 42 as described hereinabove.

The outer edge 41 of the collar 16 is provided with radially inwardly extending tabs 100 (see FIGS. 13 and 17) that engage with the detent 74, nub 76, and slot 78 provided on the neck 34 (see FIG. 10). After a user has placed the collar 16 on the neck 34 of the reservoir 12, the user rotates the collar 16 until the tabs 100 slide over the rounded nub 76 on the neck 34 and into the slot 78, upon which movement of the collar 16 is arrested due to the tabs 100 hitting the detent 74. The wings 18, which are located in opposite quadrants from the tabs 100, provide a visual indication to the consumer that the collar 16 is correctly positioned atop the reservoir 12. In other words, when the wings 18 are latitudinally aligned with the top end 26 of the reservoir 12, the collar 16 is correctly positioned atop the reservoir 12 and tabs 100 are in place within slots 78.

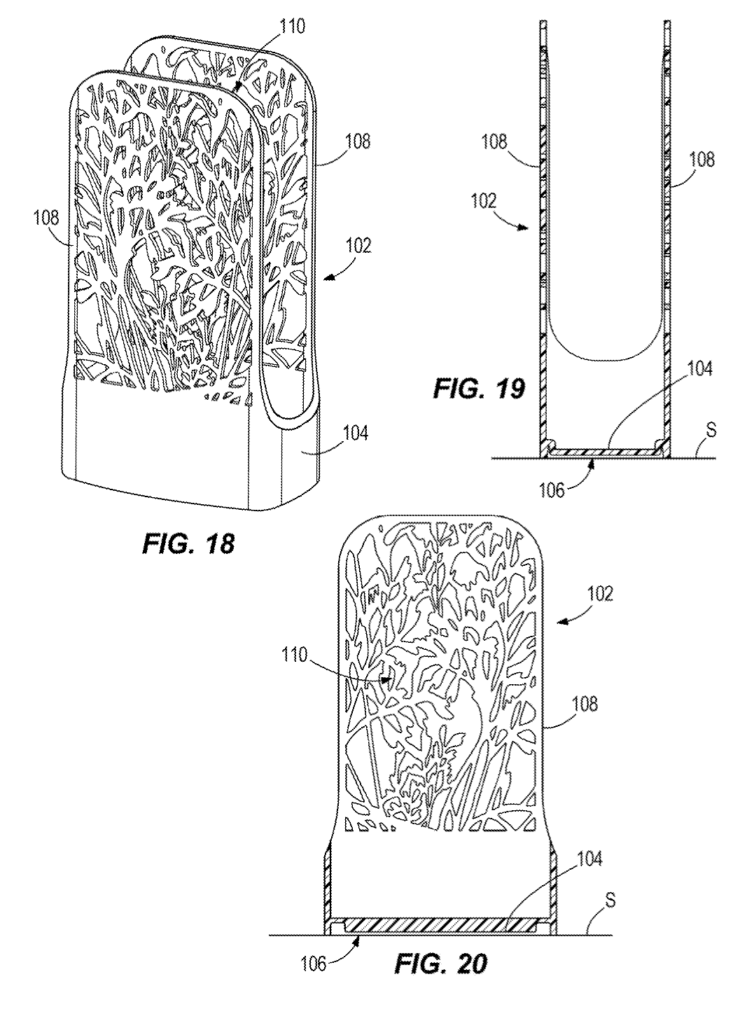

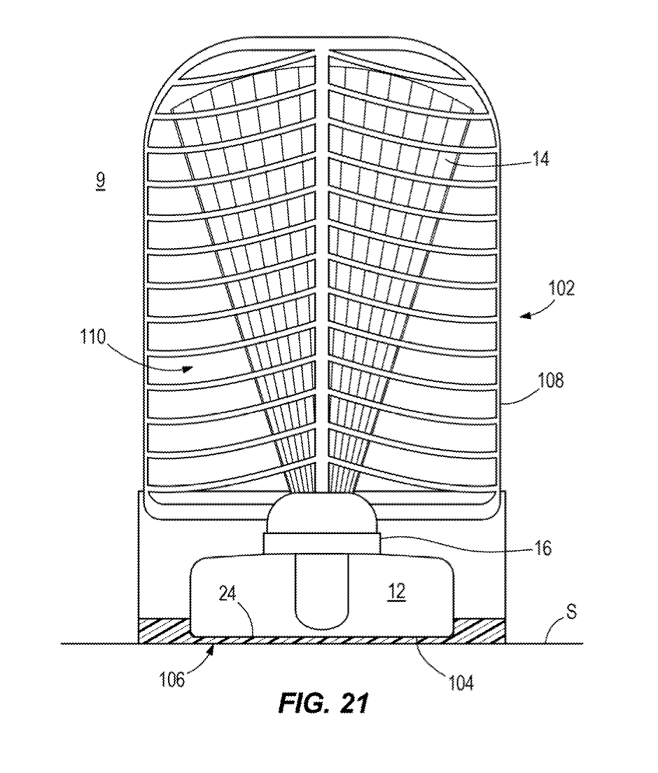

Now with reference to FIGS. 18-21, a screen 102 for surrounding the reservoir 12 and the wick 14 will be described. The screen 102 has a base 104 supporting the reservoir 12. The base 104 has a hollowed out portion 106 sized to fit the bottom end 24 of the reservoir 12. The hollowed out portion 106 may be elevated above a surface “S” upon which the screen 102 rests. The screen 102 also has side walls 108 extending upwardly from the base 104. The side walls 108 prevent a user from easily contacting the wick 14. The side walls 108 also provide aesthetic appeal to the device 10, and may have designs 110 cut through them that allow air flow from the surrounding area 9 to the wick 14.

The non-planar wick 14 of the present disclosure provides relatively greater diffusing-capable surface area in a limited space than a planar wick of the same planform surface area A (see FIG. 3). The larger the surface area of the wick 14, the more powerful the device 10 is with regards to fragrance delivery. More surface area of the wick 14 can absorb more heat from the surrounding area 9 to drive more evaporation. The device 10 of the present disclosure provides a high potential to deliver fragrance to a large room or open space. Folding and gathering the sheet of material 44 as disclosed herein above can increase the surface area of the wick 14 by 500% when compared to a planar wick of the same planform surface area. The non-planar wick 14 of the present disclosure maintains a planform surface area A (FIG. 3) that is relatively small compared to the diffusing-capable surface area (for example, W×L in FIG. 4) of the sheet of material 44. Further, providing the lower end 22 of the wick 14 with a large amount of material (due to gathering of the lower end 22 of the wick 14) increases the ability of the wick 14 to engage in capillary suction. Folding and/or gathering the wick 14 also provides structural rigidity to the wick 14, which becomes important as liquid is sucked up into the wick 14 via capillary action and the wick 14 becomes heavier. Providing the wick 14 with structural rigidity means that fewer complex plastic parts are required to hold the wick 14 in an upright position. Folding or gathering the wick 14 along a plurality of longitudinally extending bend lines 50a, 50b, 50c, etc. (FIGS. 1-5), rolling the wick 14 into a cone shape (FIGS. 6-7), folding the wick 14 into a polyhedron (FIGS. 8-9), or providing any other three-dimensional or non-planar shape to the wick 14 provides the wick 14 with structural rigidity, a relatively large dipping portion, and a relatively large diffusing-capable surface area. Various other advantages will be recognizable by those having ordinary skill in the art.

In the above description certain terms have been used for brevity, clearness and understanding. No unnecessary limitations are to be implied therefrom beyond the requirement of the prior art because such terms are used for descriptive purposes only and are intended to be broadly construed. The different devices described herein above may be used in alone or in combination with other devices. Various equivalents, alternatives and modifications are possible within the scope of the appended claims. Each limitation in the appended claims is intended to invoke interpretation under 35 USC §112, sixth paragraph only the terms “means for” or “step for” are explicitly recited in the respective limitation.

What is claimed is:

1. A device for passively diffusing a liquid to the air of a surrounding area, the device comprising:
a reservoir that holds the liquid, the reservoir having a top end with an aperture therethrough; and
a non-planar wick extending through the aperture such that a lower end of the wick remains in contact with the liquid in the reservoir and an upper end of the wick is exposed to the surrounding area;
wherein the wick is formed of a fibrous, porous sheet of material having a top edge and a bottom edge of substantially the same width; and
wherein the sheet of material is gradually gathered along a length of the sheet of material such that the upper end of the wick is less gathered than the lower end of the wick and the wick is self-supported in an upright position within the aperture.

2. The device of claim 1, wherein the reservoir comprises a main body and an upwardly extending neck, an inner surface of the neck defining the aperture.

3. The device of claim 2, further comprising a collar coupled to the neck and surrounding the wick within the aperture, so as to provide a liquid-tight connection between the wick and the reservoir.

4. The device of claim 3, further comprising a sleeve extending downwardly from a lower side of the collar and through the aperture, the sleeve having an insertion opening therethrough that receives the lower end of the wick.

5. The device of claim 4, wherein the sleeve has radially inwardly extending elastic fingers, the elastic fingers securing the lower end of the wick within the insertion opening.

6. The device of claim 4, further comprising an inner annulus and an outer annulus extending downwardly from the lower side of the collar, wherein the inner and outer annuli are located radially outwardly of the sleeve, and wherein the inner and outer annuli have a recess therebetween for insertion of the neck.

7. The device of claim 6, wherein the neck comprises an outer thread that engages with an inner thread on the outer annulus of the collar to form a threaded connection between the collar and the neck.

8. The device of claim 1, wherein the sheet of material is semi-rigid, so as to contribute to the self-supporting nature of the wick.

9. The device of claim 1, wherein the sheet of material comprises blotter paper.

10. The device of claim 1, wherein the wick absorbs the liquid at a rate ranging from about 0.2 grams per day to 2 grams per day and delivers fragrance for at least about seven days after complete consumption of the liquid in the reservoir.

11. A device for passively diffusing a liquid to the air of a surrounding area, the device comprising:
a reservoir that holds the liquid, the reservoir having a top end with an aperture therethrough;
a non-planar wick extending through the aperture such that a lower end of the wick remains in contact with the liquid in the reservoir and an upper end of the wick is exposed to the surrounding area; and
a collar coupled to the reservoir and surrounding the wick within the aperture;
wherein the collar provides a liquid-tight connection between the wick and the reservoir.

12. The device of claim 11, wherein the wick is formed from a sheet of material having a lower edge and an upper edge of substantially the same width, and wherein the sheet of material is gradually gathered along a length of the sheet of material such that the upper end of the wick is less gathered than the lower end of the wick.

13. The device of claim 12, wherein the sheet of material is pleated along a plurality of longitudinally extending bend lines, two adjacent bend lines defining a longitudinally extending wall segment of the wick, and wherein the lower end of the wick is gathered such that adjacent wall segments face one another in substantially parallel relationship and the upper end of the wick is splayed out such that adjacent wall segments extend at an angle to one another.

14. The device of claim 13, wherein the reservoir comprises a body and an upwardly extending neck, an inner surface of the neck defining the aperture.

15. The device of claim 14, further comprising a sleeve extending downwardly from a lower side of the collar and through the aperture, the sleeve having an insertion opening therethrough that receives the lower end of the wick.

16. The device of claim 15, wherein the sleeve further comprises radially inwardly extending elastic fingers, wherein the elastic fingers engage with cutouts in the lower end of the wick such that the elastic fingers secure the lower end of the wick within the insertion opening.

17. The device of claim 16, further comprising an inner annulus and an outer annulus extending downwardly from the lower side of the collar, wherein the inner and outer annuli are located radially outwardly of the sleeve, and wherein the inner and outer annuli have a recess therebetween for insertion of the neck.

18. The device of claim 17, wherein the neck comprises an outer thread that engages with an inner thread on the outer annulus of the collar to form a threaded connection between the collar and the neck.

19. The device of claim 13, wherein the longitudinally extending wall segments provide the wick with a diffusing-capable surface area that is greater than a planform surface area of the wick.

20. A device for passively diffusing a liquid to the air of a surrounding area, the device comprising:
a reservoir that holds the liquid, the reservoir having an top end with an aperture therethrough;
a non-planar wick extending through the aperture such that a lower end of the wick remains in contact with the liquid in the reservoir and an upper end of the wick is exposed to the surrounding area;
a collar coupled to the reservoir and surrounding the wick within the aperture, so as to provide a liquid-tight connection between the wick and the reservoir; and
a screen surrounding the reservoir and the wick, the screen having a base supporting the reservoir and sidewalls extending upwardly from the base and shielding the wick;
wherein the wick is pleated along a plurality of longitudinally extending bend lines, two adjacent bend lines defining a longitudinally extending wall segment of the wick; and
wherein the lower end of the wick is gathered such that adjacent wall segments face one another in substantially parallel relationship, and the upper end of the wick is splayed out such that adjacent wall segments extend at an angle to one another.
